(19) United States
(12) Patent Application Publication
(10) Pub. No.: US 2010/0303938 A1
(43) Pub. Date: Dec. 2, 2010

(54) METHOD AND PREPARATION OF HERBAL EXTRACT OF CALOTROPIS GIGANTEA FOR CANCER TREATMENT

(76) Inventor: Jacob ZACHARIA, New Delhi (IN)

Correspondence Address: GREENBERG & LIEBERMAN, LLC, 2141 WISCONSIN AVE, N.W., SUITE C-2, WASHINGTON, DC, 20007, US

(21) Appl. No.: 12/599,432

(22) PCT Filed: May 9, 2007
(86) PCT No.: PCT/IN2007/000179

§ 371 Date: Nov. 9, 2009

(30) Foreign Application Priority Data
May 9, 2007 (IN) 982/CHE/2007

(51) Int. Cl. A61K 36/61; A61K 36/27; A61P 35/00

(52) U.S. Cl. 424/769; 424/774

(57) ABSTRACT

A method of preparation of a potential herbal anti-cancer medicine from the leaves and twigs of Calotropis gigantean for prevention and treatment of all types of cancers, using fresh extract of leaves and twigs of Calotropis gigantea fortified with unrefined sugarcane sugar (jaggery). The aqueous extract of leaves and twigs of Calotropis gigantea was desiccated. The [3 H]-Thymidine growth assay was performed against cancer cell lines, WM 902-B and WM1341-D. The extract [28 mgms] was dissolved in 2 ml control at 0.5%, 0.1% DMSO. The extract is highly inhibitory at all dilutions and highly inhibitory to both the cancer cell lines.

### FIELD OF THE INVENTION

The present invention relates to methods for the treatment of cancer by herbal extract. In particular, the invention relates to methods for the treatment of cancer by a potential herbal anti-cancer medicine obtained from the extract of Calotropis gigantea R.Br.

### BACKGROUND AND PRIOR ART

Normal living cells grow in a controlled way. But cancer cells are different to normal cells. Initially, one normal cell becomes cancerous. This grows into two, then four, then eight and so on. Cancer cells may not stick together well, and are able to spread around the body. As the cancer start to develop some of the information carried in the cell's DNA becomes altered in certain ways. This altered information might tell the cell to carry on and on growing and dividing instead of stopping. There are many different types of cells in the body and any of these can become cancerous. So there are many different types of cancer. As the cancer grows, it invades the body tissues surrounding it. This is harmful to the body because it damages these surrounding normal tissues too.

The immune system has a role to play in fighting the cancer. Cancer treatments such as chemotherapy and radiotherapy can weaken the immune system. Cancers, usually, recur. Because it can come back after it seems to have been successfully treated. Cancer is both frightening to people and hard to cure. There are a number of different explanations for why cancer comes back in particular circumstances.

The Modern treatments that can cure cancer are surgery, radiotherapy and chemotherapy. Unfortunately, cancer may recur after any of these treatments. Cancer can come back after surgery because of the presence of some cancer cells left behind during the operation. Some cancer cells might have broken away from the primary cancer before the operation and spread to elsewhere in the body.

Cancer cells are killed by chemotherapy drugs because these drugs attack cells that are in the process of doubling to form new cells. Cells that are actively dividing are also more vulnerable to radiation. So radiotherapy works by killing dividing cells too. But not all the cells in a cancer will be dividing at the same time. Normal cells go into a long rest period between divisions. Cancer cells do too, although the rest period may be much shorter. Giving chemotherapy and radiotherapy in a series of treatments helps to catch as many cells dividing as possible and so kill them. Hopefully, cells that were resting when you had your first treatment (so didn't die) will be active when you have your next and so will be more likely to be killed. But it is unlikely that any cancer treatment will kill every cancer cells in the body. To do that, the treatment would have to be very strong and would have too many dangerous side effects. Doctors want to give you as much treatment as they can. But they cannot give any guarantee and safety. All treatments have a safe limit. You can only have up to a certain amount of radiotherapy to any particular part of the body. If you have more, then normal body tissue will be too damaged to repair itself. Chemotherapy drugs have their limits too. Some can cause problems with the heart, or with the liver or kidneys, if you are given too much of that one drug.

Therefore, Cancer is a menace to mankind. Its increasing incidence and mortality rates are alarming. However, no effective natural form of anti cancer medicine has not yet been discovered.

### DESCRIPTION OF THE INVENTION

The present invention relates to methods for the treatment of cancer by a potential herbal anti-cancer medicine obtained from the extract of Calotropis gigantea R.Br. “SJ-29 Compound” is the secret code given to the extract of the herb Madar or Calotropis gigantean R.Br. It belongs to Asclepiadaceae family. The extract of Calotropis gigantea R.Br is traditionally been used in the treatment of snake bites, eczema, ulcers, piles, etc. With the above background, the potential anti cancer effects of the fresh natural extract of Caltropis gigantean R.Br were studied. Another species Calotropis procera was earlier reported to have cytotoxic effect on cancers developed in mice (Smit H F, Woerdenbag H J, Singh R H, Meulenbeld G J, Labadie R P, Zwaving J H-1995). Calotropis procera is defferent from Calotropis gigantea in the coronary arrangements, shape of petals and colour of the flowers.

For in vitro experiments the aqueous extract of leaves and twigs of Calotropis gigantea R.Br. (SJ-29) was desiccated. The [3H]-Thymidine growth assay was performed against cancer cell lines, WM 902-B and WM1341-D. The extract [28 mgms] was dissolved in 2 ml control at 0.5%, 0.1% DMSO (Table 1). When compared to the control results, the extract was highly inhibitory at all dilutions.

TABLE 1
Inhibitory effects of SJ-29

Cancer Cell Lines

| Name of the | | WM 903-B | WM1341-D |
|---|---|---|---|
| Compound | Dilution | cpm(mean) | cpm(mean) |
| SJ-29 | 1:200 | 265 | 128 |
| | 1:1000 | 791 | 259 |
| | 1:5000 | 1209 | 238 |
| | 1:25000 | 2644 | 186 |
| Control | 0.50% | 18115 | 9172 |
| DMSO | 0.1% | 18484 | 4954 |
| | 0% | 15269 | 1993 |

This experiment showed that SJ-29 is highly inhibitory to both the cancer cell lines.

For cancer patients, the aqueous extract prepared was dried so as to have nearly 10% water content in it and was mixed with unrefined sugarcane sugar (jaggary) syrup. This fortified syrup was given as drops to the patients. Five drops of the medicine contained approximately 150 mgms of the dried aqueous extract. The SJ-29 preparations along with supplementary medicines were given to all kinds of cancer patients over a period of three months to two years.

For in vitro experiments, the aqueous extract of the leaves and twigs of Calotropis gigantea R.Br. (SJ-29) was dessicated. This extract dissolved in DMSO was tested against cancer cell lines, WM 903-B and WM-1341-D using [3H]-Thymidine growth assay. DMSO was used as control. In the preliminary studies, SJ-29 showed higher inhibition of the cancer cell lines.

The Fresh extract of leaves and twigs of Calotropis gigantea R.Br. (SJ-29) fortified with unrefined sugarcane sugar (jaggery), (Prepared in traditional Ayurvedic Style) when administered in various categories of volunteer patients gave astonishing results of cure, relief of pain and suffering (palliative) and increased expectancy of life span. The medicine is safe, leaving no side effects on administration. Also this unique treatment is less expensive.

Its (SJ-29) anti-cancer activity against human cells lines (WM 902 B WM-1341-D WM-852) was noticed and many repeated testing with many other melanoma cell lines were also done. The results were excellent. The plant extract dissolved in DMSO at various concentrations were used for testing. It was inhibitory even at 0.1 microgram/ml and almost complete inhibition was noticed with 2.5 microgram/ml. The thymidine syntheses for the multiplication of cancer cells are inhibited by SJ-29. Further growth and multiplication is not possible. The beauty of SJ-29 is that in human patients it acts selectively only on cancer cells. It is very safe and has absolutely no side effects on patients.

### Preparation and Administration of Special Drop Concentrate

Preparation SJ-29 produced in drops, tablets, injections, syrups and ointment. Special drop concentrate prepared is easily administered. The optimum dose of Special Drops given to patients was approximately 100 u/mg. each three times daily. Special Ointment prepared could be mixed with additives like clove oil, creams for getting the desired results.

The Special drop concentrate has become the medicine of choice for cancer treatment to many patients, all over the world. Supplementary medicines are inevitable for easy recovery. All the vital organs start dysfunction from the onset of the disease. So, revitalization of all organs is to be done simultaneously with the supplementary medicines.

### Treatment

Fresh extract of leaves and twigs of Calotropis gigantea R.Br (SJ-29), (tablet, drops, injections, syrup and ointment) prepared was administered to volunteer patients who had no hope of survival. The results were amazing. The inventor Dr. Z. Jacob is a well-known Traditional Ayurvedic Medical practitioner, Medical Microbiologist, Biotechnologist and an Oncologist and currently the founder Director of AAMRC, (Athulya Ayurvedic and Medical Research Centre) Calicut. Dr. Jacob, after completion of his M.Sc. (Botany) from Kerala University in 1976 and M.C.T. from Calicut University in 1977, joined the prestigious Central Drug Research Institute, Lucknow and researched on studies of some fungi of medical importance. The studies encompassed mainly some superficial, systemic and ophthalmic infections and formulation and treatment with newer medicines. The above work was conducted in association with the King's George Medical College and the Army Base Hospital, Lucknow and the Kanpur University awarded the doctoral degree in 1983. During 1983-'91 he was associated with the Central Food Technological Research Institute, Mysore, worked in various capacities for the development of new biotechnological processes for the production of SCO, biosurfactants and polyalcohols. His mission to USA (1992-'93) was a turning point in his career when he worked with Prof. Cy Stein at the Department of Medicine, Division of Oncology, College of Physicians and Surgeons of Columbia University, New York, to research on immunological and genetical aspects related to the development of antisensors as anticancer medicines. He coined his knowledge of modern medicine and oncology with Ayurvedic medicine and developed a unique style of Ayurvedic treatment, which gave excellent and unimaginable results. It is worthwhile to quote few instances (curing of paralyzed and vision impaired Brain Tumor patients, curing of rarely seen cases of Spindle Cell Sarcoma, Chondrosarcoma and other Sarcomas, regaining of normalcy with a single drop of medicine daily in a five year old child, stage III and IV breast, Ovarian and Cervix cancer patients, Epithelioma and Histocytoma patients (once advised for amputation of their limbs) now working like normal individual, listening to speeches of speechless (vocal cord affected) patients, survival of Non-Hodgkin lymphoma, lung cancer and Prostrate cancer patients, increasing the life expectancy of lymphoma patients, obtaining normalcy in Wilm tumor, Ewing Sarcoma, Neuroblastoma, Astrocytoma Meningioma patients, getting pain relief and subsequent frequent deaths of terminal patients with Pancreatic or Hepatobilary or Renal Cancer). Continued medication in calculated dosages by the clock and by the ladder and supplementary symptomatic treatment gave definite relief, even for certain patients (having cancer advanced stages) whose vital organs lost functioning or destroyed. Effective cure without any recurrence in a reasonable period of time has been observed in all categories of cancer occurring at different sites viz. Breast, Colon, Germ cell, Lung, Oesophazus, Prostrate and other forms like Leukamia, Lymphoma, Multiple myeloma, Oesteosarcoma, Neuroblastomas, Meningoima, and Gestation period, etc.

### EXAMPLES

### List of Few Challenging Cases Recovered, Category Wise for Reference as on 15 Mar. 2007

1. Patient Mr. P. C (Calicut) suffering from recurring EPITHELIOMA (squamous cell type) of leg. The lesion was excised before and treated with SJ-29 in 1996. No symptoms of the disease occurred since 1996 and live and work like normal individual.
2. Patient Mrs. S V (Ernakulam) suffering from PAPILLARY THYROID CANCER while at 3^{rd} month of pregnancy of her first child in August 2001 was advised for abortion and chemotherapy and radiation therapy. After treatment at AAMRC saved the foetus and mother, with out any symptoms of the cancer in the newborn baby and mother. In 2002 she gave birth to another normal child with out any signs of the disease.
3. Patient Mrs. DK (Calicut) suffered CHONDROSARCOMA and successfully cured in 2001 and no recurrence since then noticed.
4. Patient Mr. K (Ernakulam) diagnosed with recurring PAROTID CANCER treated at AAMRC in June '05 and now lives and works like a normal man.
5. Patient Mrs. V (Kottayam) treated for recurring CANCER LARYNX was cured in January 99 and did not get any recurrence so far.
6. Patient Mrs. S (Malappuram) had recurring CANCER CERVIX and successfully cured in 1997. Now continuing her work as Teacher in a school.
7. Patient Mrs. C K S (Calicut) recurring COLON CANCER with bleeding was cured since May 2001.
8. Patient Mr. R (Calicut) had recurring EWINGS TUMOR and cured completely in 1997. Mr. R is now grown up and married.
9. Patient Mr. N. K (Germany) suffering from LEUKOPLAKIA for more than 10 years cured in one weeks time in September '05. No recurrence noticed since them.
10. Patient Mr. P A R (Calicut) recurring ASTROCYTOMA in terminal coma stage was cured completely in 1990.
11. Patient Mr. P M J (Wayanad) had recurring ADENOCARCINOMA OF THYROID AND VOCAL CORD and cured completely in October 1997.
12. Patient Mr. M G (Gudallur) diagnosed with SQUAMOUS CELL CARCINOMA OF CHEEK was cured in twelve days treatment in 1998.
13. Patient B K (Palghat) recurring OSTEOSACCOMA of lower mandible in 1997 and treated and completely cured.
14. Patient Mrs. S G (Calicut) recurring INTRADUCT BREAST CANCER in 2003 and completely cured and reported no recurrence so far.
15. Patient C K (Quilon) was suffering from recurring LEIOMYOSARCOMA in October '03 and no complaint of the disease is reported so far.
16. Patient Mr. V. C. J (New Delhi) had PROSTRATE CANCER and treated and cured since '05. The PSA values are within normal limits.
17. Patient Mr. A. V. H (USA) had recurring type of PROSTRATE CANCER with metastasis was cured in July '04.
18. Patient L (Italy) was suffering form recurring UTERUS CANCER WITH METASTASIS and remarkable recovery in 2003. Now live and work like a normal individual.
19. Patient A K S (Germany) had recurring OVARIAN AND BREAST CANCER WITH METASTASIS was treated and cured in February '05. All the markers are in normal limits and lead a normal life.
20. Patient Mrs. K. K (Hyderabad) had CANCER LUNG WITH METASTASIS TO BRAIN affecting the eye vision in December '06. Three months medication and further testing reported that she is free of the disease. Now the vision is normal.
21. Mr. S P (Malappuram) had RECTAL CANCER in the advanced stage was treated in December '06 and no symptoms of the disease observed after 2 months of medication and lead a normal life
22. Mrs. N. T (Japan) had recurring BREAST CANCER WITH METASTASIS took treatment at AAMRC during November '06. When tested, after 5 months, it was reported that her CEA, CA-15-3, NCCST439 Values are within normal limits. Now she leads a normal life.
23. Mr. N. P (Palghat) was suffering from OESOPHAGAL CANCER with total dysphagia for solids and liquids for nearly one year was treated at AAMRC during September '06 and got completely cured.
24. Mr. R V S (Bombay) suffering from recurring HODKINS LYMPHOMA affecting the eye vision was completely cured after taking treatment at AAMRC during August '06.
25. Mrs. R. R (Bangalore) had recurring NON HODKINS LYMPHOMA with metastasis and had treatment since July '06 and she is normal now.
26. Mrs. H. G (Belgium) had recurring BREAST CANCER WITH METASTASIS was on AAMRC treatment in March 2006. When report last came in December '06 she was normal and the markers were in normal limits.
27. Mr. G I (Russia) had HEPATITIS B (HEPATOMA) was treated at AAMRC in December '05 and completely free from the liver disease.
28. Baby S (Hungary) had NEUROBLASTOMA and she successfully cured in February 2006. No signs of the disease or any recurrence noticed after wards.
29. Master N. T. P (Calicut) suffering form ACQUIRED APLASTIC ANEMIA is on treatment form AAMRC with effect from January 2003. Improved levels of red blood cells, platelets and goes to school like a normal child.
30. Master M. V (Pune) suffering form recurring ACUTE LYMPHOBLASTIC LEUKAEMIA was effectively treated at AAMRC during August '05.

AAMRC is entertaining all types of cancer patients. They are treated as inpatients or out patients. AAMRC recommend surgical intervention to patients as and when required. And don't recommend chemotherapy and radiation therapy. Cent percent success rate is possible in patients who have not received any other types of treatment like chemotherapy (CT) and radiation therapy (RT) (for e.g. Squamous Cell Carcinoma and adenocarcinoma patients). For other types of cancers which affect the ovary, prostrate etc and who received CT and RT earlier, the after effects have to be nullified and anticancer treatment have to be performed simultaneously. There is another category where the patients go for surgical interventions elsewhere and further anticancer treatments are carried out at AAMRC. The success rate in advanced stage is more than 50%. We are proud to say that many such cases have recovered miraculously from cancer.

This medicine can also be taken as a prophylactic agent and many are being used now days. The biggest advantage is that it has no side effects. The aqueous extract of SJ-29 was desiccated so as to obtain 10% water content and mixed with jaggery syrup and this was used as medicine to administer in all types of cancer patients for over different periods of time (3 months to 2 years). The medicine was administered as drops, one drop a day to patients below 5 years, 2 drops a day to patients below 10 yeas, 3 drops a day to patients below 15 years and 5 drops a day to patients below 20 years and 10 to 15 drops a day to patients above 20 years of age. According to the symptoms, besides SJ-29, all the patients were administered with supplementary ayurvedic medicines and therapies. Amazing results of cure, relief of pain and sufferings and extension of life span were noticed in all the cancer patients. Most of the patients were in their terminal stages and subjected to surgery, chemotherapy and radiation therapy, some of the patients received no other treatment at all. Those who had prostrate cancer; the prostrate specific antigen values became normal. In ovarian or uterus cancer patients the CEA came down to normal limits. In breast cancer patients CEA, CA-15-3 and NCCST439 also fell to normal levels. In many hepatitis cancer patients, their healths become normal after a medication of 3 to 6 months. There were instances of curing of initial stage squamous cell carcinoma of the cheek in a week's time, curing of blurred vision and incomprehensive speech in a Hodgkins Lymphoma patient and in a patient of lung cancer with metastasis in 14 to 21 days, detection of thyroid cancer in a patient at third month pregnancy and giving cure and normal delivery of healthy child, curing brain cancer in a less than one year old child, etc. Similarly, there are many patients of breast cancer, sarcoma, leiomyosarcoma, colorectal cancer, oesophagal cancer, pancreatic cancer, kidneys were also benefited.

I claim:

1. A method of preparation of a potential herbal anti-cancer medicine from the leaves and twigs of Calotropis gigantea for prevention and treatment of all types of cancers, using fresh extract of leaves and twigs of Calotropis gigantea fortified with unrefined sugarcane sugar (jaggery).

2. A method of preparation of extract from the leaves and twigs of Calotropis gigantea for prevention and treatment of cancers. The aqueous extract of leaves and twigs of Calotropis gigantea was desiccated. The [3H]-Thymidine growth assay was performed against cancer cell lines, WM 902-B and WM1341-D. The extract [28 mgms] was dissolved in 2 ml control at 0.5%, 0.1% DMSO. The extract is highly inhibitory at all dilutions and highly inhibitory to both the cancer cell lines

3. A method of preparation of extract from the leaves and twigs of Calotropis gigantean claimed in claim 2 wherein Inhibitory effects of Calotropis gigantean is
Cancer Cell Lines

| | | WM 903-B | WM1341-D |
|---|---|---|---|
| | Dilution | cpm(mean) | cpm(mean) |
| | 1:200 | 265 | 128 |
| | 1:1000 | 791 | 259 |
| | 1:5000 | 1209 | 238 |
| | 1:25000 | 2644 | 186 |
| Control | 0.50% | 18115 | 9172 |
| DMSO | 0.1% | 18484 | 4954 |
| | 0% | 15269 | 1993 |

4. A method of preparation of extract from the leaves and twigs of Calotropis gigantean claimed in claim 2 wherein, the aqueous extract prepared was dried so as to have nearly 10% water content in it.

5. A method of preparation of extract from the leaves and twigs of Calotropis gigantean claimed in claim 2 wherein five drops of the medicine contained approximately 150 mgms of the dried aqueous extract

6. A method of preparation of extract from the leaves and twigs of Calotropis gigantean claimed in claim 2 wherein, the thymidine syntheses for the multiplication of cancer cells are inhibited.

7. A method of preparation of extract from the leaves and twigs of Calotropis gigantean claimed in claim 2 wherein, further growth and multiplication of the cancer cells will be inhibited

8. A method of preparation of extract from the leaves and twigs of Calotropis gigantean claimed in claim 2 wherein, that in human patients it acts selectively only on cancer cells.

9. A method of preparation of extract from the leaves and twigs of Calotropis gigantean claimed in claim 2 wherein, the suspension has no side effects.

10. A method of preparation of extract from the leaves and twigs of Calotropis gigantean claimed in claim 2 wherein, that may be produced as drops, tablets, injections, syrups and ointments.

11. A method of preparation of extract from the leaves and twigs of Calotropis gigantean as a mode of ointment claimed in claim 10 wherein special ointment prepared could be mixed with additives like clove oil, creams for getting the desired results.
